(19) United States
(12) Patent Application Publication
(10) Pub. No.: US 2026/0137556 A1
(43) Pub. Date: May 21, 2026

(54) WIRELESS INTELLIGENTLY CONTROLLED SYSTEM FOR INTRAVESICAL TUMOR HYPERTHERMIA

(71) Applicants: Zhihui Zou, Hefei (CN); THE FIRST AFFILIATED HOSPITAL OF ANHUI MEDICAL UNIVERSITY, Hefei (CN)

(72) Inventors: Zhihui Zou, Hefei (CN); Siyuan Xia, Hefei (CN); Zhengyang Wu, Hefei (CN); Shuchen Liu, Hefei (CN); Kaixuan Zhang, Hefei (CN); Han Yang, Hefei (CN); Qi Zhang, Hefei (CN); Zicheng Li, Hefei (CN); Qi 'ao Jing, Hefei (CN); Jiaqing Zou, Hefei (CN); Qinglan Zheng, Hefei (CN); Jiaying Song, Hefei (CN); Yongtao Hu, Hefei (CN); Yu He, Hefei (CN); Keke Cai, Hefei (CN); Ligang Zhang, Hefei (CN); Aiping Wang, Hefei (CN); Zongyao Hao, Hefei (CN); Chaozhao Liang, Hefei (CN)

(21) Appl. No.: 19/389,184

(22) Filed: Nov. 14, 2025

(30) Foreign Application Priority Data
Nov. 15, 2024 (CN) 2024116360496

(51) Int. Cl. A61F 7/12; A61B 1/00; A61B 1/04; H02J 50/10

(52) U.S. Cl. CPC A61F 7/12; A61B 1/00016; A61B 1/041; H02J 50/10; A61F 2007/126

(57) ABSTRACT

includes an implantable intravesical floating hyperthermia device and an external pulse heating apparatus. The floating device includes a sealed non-metallic housing, a metal coil configured to inductively couple with an alternating magnetic field generated outside the human body to generate heat for tumor hyperthermia, a temperature sensor, a wireless transmission module, and a power supply module. The pulse heating apparatus includes at least one magnetic field generation module and a control module that controls the heating temperature according to the temperature signal from the floating device. The system supports multiple, timed, and temperature-controlled hyperthermia sessions with a single transurethral insertion, reducing patient trauma and enabling outpatient self-management.

### FIELD OF THE INVENTION

The present invention relates to the technical field of medical devices, and more particularly to a wireless intelligently controlled system for intravesical tumor hyperthermia, a placement rod and a wireless tumor hyperthermia assembly.

### BACKGROUND OF THE INVENTION

Tumor hyperthermia generally refers to a class of treatment methods that use heating to treat tumors. It has become the fifth major therapy following surgery, radiotherapy, chemotherapy, and immunotherapy, serving as a new and effective means for treating tumors and is known as “minimally invasive.” Among these, intravesical tumors, due to their cavity structure suitable for heating, provide an anatomical basis for implementing tumor hyperthermia.

Currently, there are two methods for heating intravesical tumors: (1) circulating hot saline solution through a three-lumen catheter connected to the bladder inside the body and a heat source outside the body; and (2) implanting a metal heating wire into the bladder, connected via wires through the urethra, to heat the metal wire. Both methods require repeated invasive urethral procedures during periodic intravesical tumor hyperthermia sessions. Such repetitive catheterization operations are highly prone to postoperative complications such as infection, bleeding, and urethral strictures, causing significant physiological and psychological trauma to patients.

### SUMMARY OF THE INVENTION

Therefore, the object of the present invention is to provide a wireless intelligently controlled system, a placement rod and a wireless tumor hyperthermia assembly, aiming to address the issue in existing tumor hyperthermia technology where instruments need to be repeatedly implanted into the human body for each session, causing immense suffering to patients.

The present invention provides a wireless intelligently controlled system for intravesical tumor hyperthermia. The system includes a intravesical floating hyperthermia device and a pulse heating apparatus. The floating device includes a hermetically sealed housing made of a non-metallic material, a temperature sensor disposed within the housing and configured to detect a temperature within the housing and generate a corresponding temperature signal, a wireless transmission module disposed within the housing and configured to wirelessly transmit the temperature signal externally, and a power supply module disposed within the housing and configured to supply power to the temperature sensor and the wireless transmission module. The pulse heating apparatus includes at least one magnetic field generation module configured to emit the alternating magnetic field toward the metal coil and a control module configured to receive the temperature signal and control a heating temperature of the metal coil within a set temperature threshold range based on the temperature signal.

In the system of the present invention, the interior of the housing is divided into a cavity and a gas chamber configured to provide buoyancy to float the entire floating device within a urine; and wherein the metal coil, the temperature sensor, the wireless transmission module, and the power supply module are all located within the cavity.

In the system of the present invention, the cavity is filled with a thermally conductive urine configured to uniformly transfer heat generated by the metal coil to a surface of the housing.

In the system of the present invention, the floating device further includes a camera module disposed on the housing and configured to capture images and video external to the housing; and wherein the wireless transmission module is further configured to wirelessly transmit the images and the video to an external receiving device; and wherein the power supply module is further configured to supply power to the camera module.

In the system of the present invention, the pulse heating apparatus further includes a fixing member configured to be secured to the human body, the fixing member is structured as a belt, and two magnetic field generation modules are connected to both front and rear sides of the fixing member.

In the system of the present invention, the control module is configured to compare a value of the temperature signal with the temperature threshold range and:
control at least one magnetic field generation module to increase an intensity of the alternating magnetic field if the value is less than a minimum value of the temperature threshold range;
control at least one magnetic field generation module to decrease the intensity of the alternating magnetic field if the value is greater than a maximum value of the temperature threshold range; and
control at least one magnetic field generation module to maintain the intensity of the alternating magnetic field if the value is within the temperature threshold range

In the system of the present invention, the pulse heating apparatus further includes a portable power source configured to supply power to the at least one magnetic field generation module and the control module.

In the system of the present invention, an outer surface of the housing is coated with an antibacterial, hydrophilic, anti-adhesion coating.

In the system of the present invention, the camera module includes a camera unit and a light source.

In the system of the present invention, the thermally conductive urine is biocompatible heat transfer fluid.

In the system of the present invention, the gas chamber is filled with air or an inert gas.

In the system of the present invention, the housing is provided with a docking slot at one end, and a magnet is connected within the docking slot.

In the system of the present invention, the control module is further configured to set the temperature threshold range via a signal connection with an external mobile device.

In the system of the present invention, the metal coil is formed of copper wire wound into a coil structure.

In the system of the present invention, the temperature sensor, the wireless transmission module, and the power supply module are integrated together to form an integrated unit within the housing.

In the system of the present invention, the floating device is configured such that an average density of an end of the floating device proximate to the magnet is lower than an average density of an opposing end, thereby causing the end with the magnet to orient downward when the floating device is floating in a urine to facilitate docking.

In the system of the present invention, the power supply module includes a lithium battery.

The present invention also provides a placement rod for a intravesical floating hyperthermia device as described above; the placement rod includes a rod body and an electromagnet disposed at one end of the rod body; the electromagnet is configured to, when energized, magnetically engage with a magnet disposed on the intravesical floating hyperthermia device to detachably secure the floating device.

In the placement rod of the present invention, an end of the rod body is provided with a docking head matching a docking slot of the housing of the floating device, and wherein the electromagnet is disposed on the docking head.

The present invention also provides a wireless tumor hyperthermia assembly; the assembly includes a tumor pulse hyperthermia system and a placement rod as described above.

To solve the above problem, the intravesical floating hyperthermia device after being implanted into the corresponding urinary bladder cavity, allows for multiple, timed, and temperature-controlled tumor hyperthermia sessions as needed, while also enabling full endoscopic monitoring within the cavity via the built-in camera. Upon completion of the hyperthermia or endoscopic examination, the intravesical floating hyperthermia device is simply removed from the urinary bladder cavity. The entire treatment process involves only two transurethral insertion procedures. Compared to existing tumor hyperthermia methods, this avoids repeated, long-term, and invasive transurethral insertion procedures and endoscopic examinations, thereby significantly reducing the immense suffering inflicted on patients by repetitive surgical operations and intracavitary endoscopy.

Existing tumor hyperthermia requires operation by professional medical staff and specialized equipment in a hospital setting. The disposable, implantable wireless tumor intravesical floating hyperthermia device designed in the present invention only requires hospital visits for the transurethral insertion and removal procedures. The remaining hyperthermia process can be performed anywhere, anytime, or on schedule by the patient themselves, thus effectively conserving medical resources and reducing treatment costs for patients.

### BRIEF DESCRIPTION OF THE DRAWINGS

The above objects and advantages of the present invention will become more readily apparent to those ordinarily skilled in the art after reviewing the following detailed description and accompanying drawings, in which:

FIG. 1 is a structural schematic diagram of a wireless tumor intravesical floating hyperthermia device according to one embodiment.

FIG. 2 is a structural schematic diagram of a wireless tumor intravesical floating hyperthermia device according to another embodiment.

FIG. 3 is a schematic diagram illustrating the use of a wireless tumor hyperthermia system according to one embodiment.

FIG. 4 is a structural schematic diagram of a placement rod according to one embodiment.

FIG. 5 is a schematic diagram illustrating the engagement between a wireless tumor intravesical floating hyperthermia device and a placement rod according to one embodiment.

### DETAILED DESCRIPTION OF PREFERRED EMBODIMENTS

The technical solutions in the embodiments of the present invention will be clearly and completely described below with reference to the accompanying drawings in the embodiments of the present invention. It is apparent that the described embodiments are only a part of the embodiments of the present invention, rather than all of them. All other embodiments obtained by those of ordinary skill in the art based on the embodiments of the present invention without creative efforts shall fall within the protection scope of the present invention.

It should be noted that when a component is referred to as being “mounted on” another component, it can be directly on the other component or there may be an intervening component. When a component is considered to be “disposed on” another component, it can be directly disposed on the other component or there may be an intervening component. When a component is considered to be “fixed to” another component, it can be directly fixed to the other component or there may be an intervening component.

It is to be understood that terms such as “length,” “width,” “upper,” “lower,” “front,” “rear,” “left,” “right,” “vertical,” “horizontal,” “top,” “bottom,” “inner,” “outer,” and the like indicate orientations or positional relationships based on those shown in the drawings. They are used only for convenience in describing the present application and simplifying the description, and do not indicate or imply that the referred apparatus or elements must have a specific orientation, be constructed, or operate in a specific orientation. Therefore, they should not be construed as limiting the present application.

Unless otherwise defined, all technical and scientific terms used herein have the same meanings as commonly understood by one of ordinary skill in the technical field to which this invention belongs. The terms used in the description of the present invention herein are for the purpose of describing specific embodiments only and are not intended to limit the invention. The term “or/and” as used herein includes any and all combinations of one or more of the associated listed items.

### Embodiment 1

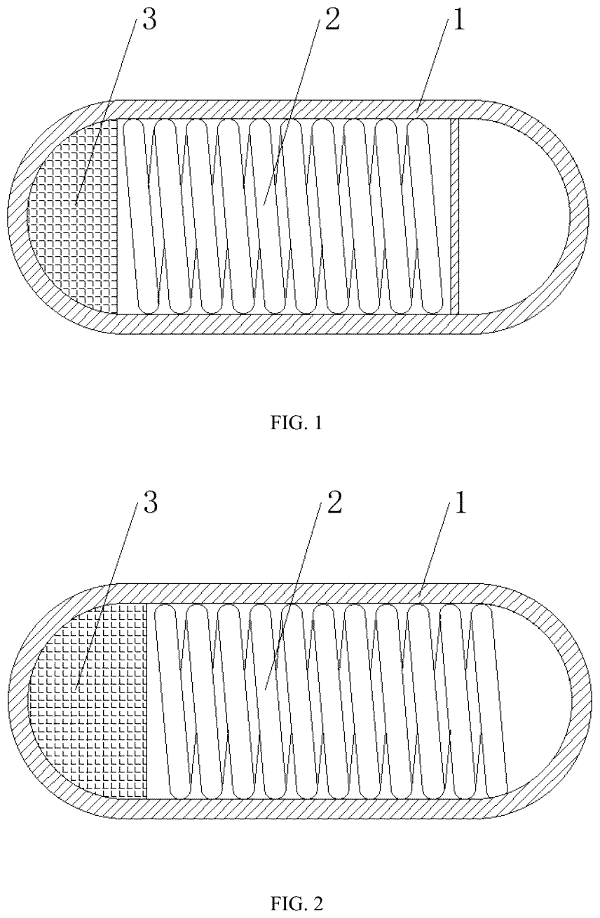

Referring to FIG. 1, a intravesical floating hyperthermia device is shown as an embodiment. The intravesical floating hyperthermia device configured to be implanted into a urinary bladder cavity and to be remotely controlled from outside the human body to perform hyperthermia on a tumor. The floating device includes a housing 1, a metal coil 2, a temperature sensor, a wireless transmission module, and a power supply module. The overall shape of the housing 1 can be set as a cylinder, which is sealed as a whole with rounded and smoothed at the edges to prevent damage to tissues when inserted into the human body. The housing 1 is hermetically sealed and made of a non-metallic material, such as a polymer, with edges rounded and smoothed to avoid tissue damage during transurethral insertion. The outer surface may be coated with an antibacterial, hydrophilic, and anti-adhesion coating to reduce irritation. The housing 1 has a diameter not greater than 7 mm and an overall length not greater than 18 mm.

The interior of the housing 1 is divided into a cavity and a gas chamber configured to provide buoyancy to float the entire floating device within a urine. The size of the gas chamber, i.e., the magnitude of buoyancy, is designed such that the entire floating device is suspended in the urine, such as urine within the bladder, to prevent it from settling on a specific area of the bladder wall causing irritation and failing to achieve the required heating temperature at the tumor site. The gas chamber is filled with air or an inert gas to provide buoyancy, enabling the floating device to float in urines such as urine in the bladder.

The metal coil 2, temperature sensor, wireless transmission module, and power supply module are all located within the cavity of the housing 1. The cavity may be filled with a thermally conductive urine such as, but not limited to, biocompatible heat transfer fluid, configured to uniformly transfer heat generated by the metal coil 2 to the surface of the housing 1, while also providing electrical insulation.

The metal coil 2 is configured to inductively couple with an alternating magnetic field generated outside the human body to generate heat for tumor hyperthermia. The generated heat is dissipated to the outside through the housing 1, thereby providing hyperthermia to the inserted area. The metal coil 2 is formed of copper wire wound into a coil structure. The temperature sensor is configured to detect a temperature within the housing 1 and generate a corresponding temperature signal. The wireless transmission module is configured to wirelessly transmit the temperature signal externally. The power supply module is configured to supply power to the temperature sensor and the wireless transmission module. The power supply module includes a lithium battery, is compact and solely powers the temperature sensor and wireless transmission module. The temperature sensor, wireless transmission module, and power supply module are integrated together to form an integrated unit within the housing 1, and they may be integrated into a single unit 3.

### Embodiment 2

Referring to FIG. 2, this embodiment differs from Embodiments 1 in the housing 1. The housing 1 may omit a separate gas chamber. The cavity itself is expanded to provide buoyancy, allowing the floating device to float in urine. In this design, the cavity is not filled with a thermally conductive urine, and the sealed cavity volume is appropriately enlarged. While housing the integrated unit, the cavity also serves the function of the gas chamber, enabling the intravesical floating hyperthermia device to float in the urine. In a non-buoyant design, the floating device may be miniaturized for use in body cavities without urine, such as the intestinal or female reproductive tracts.

In another design, the volume of the cavity is not enlarged, causing the intravesical floating hyperthermia device to lose its floating capability. However, this further reduces the overall volume of the floating device, making it more suitable for use in body parts without a floating scenario, such as the human intestinal tract or female reproductive tract.

### Embodiment 3

This embodiment differs from Embodiments 1 or 2 in the modules. The floating device may further include a camera module disposed on the housing 1 and configured to capture images and video external to the housing 1. The camera module includes a camera unit and a light source, disposed on the housing 1. The camera unit may include a CCD or CMOS sensor. The number and installation positions of the camera units are not limited and can be designed according to the needs of imaging and endoscopic examination to ensure the observation range meets the required standards. The CCD sensor is capable of converting video into digital signals.

The camera module captures images and video external to the housing 1, and the wireless transmission module transmits this data to an external receiving device. The external receiving device includes a display screen that converts the received digital signals back into video, ultimately displaying the temperature detected by the intravesical floating hyperthermia device along with the video information. The power supply module also powers the camera module. The light source is configured to illuminate the shooting direction of the camera module.

### Embodiment 4

This embodiment differs from Embodiments 1 in the housing 1. The housing 1 may include a docking slot at one end, with a magnet 4 embedded within. This facilitates secure engagement with a placement rod for transurethral insertion and retrieval. The floating device is configured such that an average density of an end of the floating device proximate to the magnet 4 is lower than an average density of an opposing end, thereby causing the end with the magnet 4 to orient downward when the floating device is floating in a urine to facilitate docking.

### Embodiment 5

A pulse heating apparatus is shown as an embodiment. The pulse heating apparatus includes a fixing member 5, at least one magnetic field generation module, and a control module. The at least one magnetic field generation module is configured to emit the alternating magnetic field toward the metal coil 2, causing the metal coil 2 to be inductively heated. The magnetic field generation module includes a transmitting coil and a power supply unit. The number of the magnetic field generation modules can be two or more. The control module is fixedly connected to fixing member 5 and configured to receive the temperature signal and control a heating temperature of the metal coil 2 within a set temperature threshold range based on the temperature signal. The control module is further configured to set the temperature threshold range via a signal connection with an external mobile device. Alternatively, the temperature threshold range may be set via an adjustment switch on the belt-shaped fixing member 5.

The fixing member 5 is configured to be secured to the human body, the fixing member 5 is structured as a belt. The belt may have magnetic field generation modules on both front and rear sides to enhance magnetic field intensity. The two magnetic field generation modules are connected to both front and rear sides of the fixing member 5. In use, the belt-shaped fixing member 5 is convenient to wear on the human body and can closely adhere to the skin, keeping the distance between the magnetic field generation module and the implanted intravesical floating hyperthermia device relatively consistent during each therapy session and reducing magnetic field attenuation.

The pulse heating apparatus further includes a portable power source. The portable power source (e.g., a mobile power bank) may supply power to the magnetic field generation modules and control module. The power supply unit of the magnetic field generation module can be detachably connected to the portable power source, facilitating easy power supply anytime, anywhere and patient removal and mobility.

The control module compares the temperature signal value with the temperature threshold range and: (1) control at least one magnetic field generation module to increase an intensity of the alternating magnetic field if the value is less than a minimum value of the temperature threshold range; (2) control at least one magnetic field generation module to decrease the intensity of the alternating magnetic field if the value is greater than a maximum value of the temperature threshold range; and (3) control at least one magnetic field generation module to maintain the intensity of the alternating magnetic field if the value is within the temperature threshold range.

The temperature threshold range may be set via a mobile device connected to the control module through Bluetooth or other wireless means. According to Faraday's law of electromagnetic induction, parameters such as the number of coil windings, current intensity, and the distance between the two coils are known. Thus, the heat generated by the metal coil 2 can be controlled by adjusting the current intensity of the magnetic field generating module on the fixing member 5.

### Embodiment 6

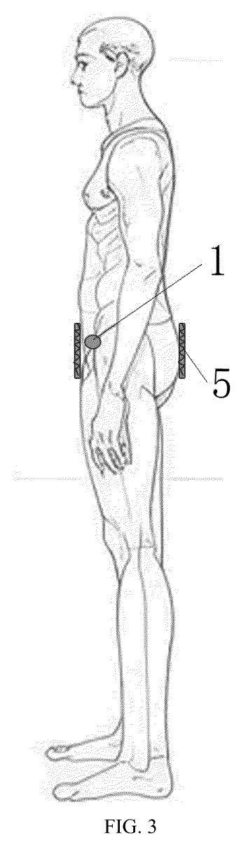

Referring to FIG. 3, a wireless intelligently controlled system for intravesical tumor hyperthermia is shown as an embodiment. The system includes the intravesical floating hyperthermia device as described in the above embodiments and the pulse heating apparatus as described in the embodiment 5. The floating device is implanted into the body cavity, and the belt-shaped fixing member 5 is worn on the body.

The magnetic field generation module emits an alternating magnetic field, inducing current in the metal coil 2 to generate heat. The temperature sensor monitors the temperature in real-time, ensuring optimal hyperthermia conditions (e.g., 42.5° C. for at least 45 minutes). The general hyperthermia requires a heating temperature of about 42.5° C. with continuous maintenance for no less than 45 minutes. Excessively high temperature can easily damage normal human cells, while excessively low temperature fails to achieve the ideal tumor treatment effect.

Based on the phenomenon of electromagnetic induction, the alternating electromagnetic field generated by the transmitting coil of the magnetic field generation module couples with the metal coil 2 via inductive coupling. When the metal coil 2 is in proximity to the transmitting coil, it is subjected to electromagnetic induction, inducing a corresponding current, which in turn causes the metal coil 2 to heat up and generate thermal energy. Furthermore, according to Faraday's law of electromagnetic induction, with known parameters such as the number of coil windings, the current intensity in the transmitting coil, and the distance between the two coils, the heat generated by the metal coil 2 can be controlled by adjusting the current intensity of the magnetic field generating module on the fixing member 5.

During use, the belt-shaped fixing member 5 is convenient to wear on the human body and can closely adhere to the skin. This ensures that the distance between the transmitting coil and the metal coil 2 remain relatively consistent during each hyperthermia session, while also positioning the magnetic field generation module as close as possible to the implanted intravesical floating hyperthermia device, thereby reducing magnetic field attenuation.

### Embodiment 7

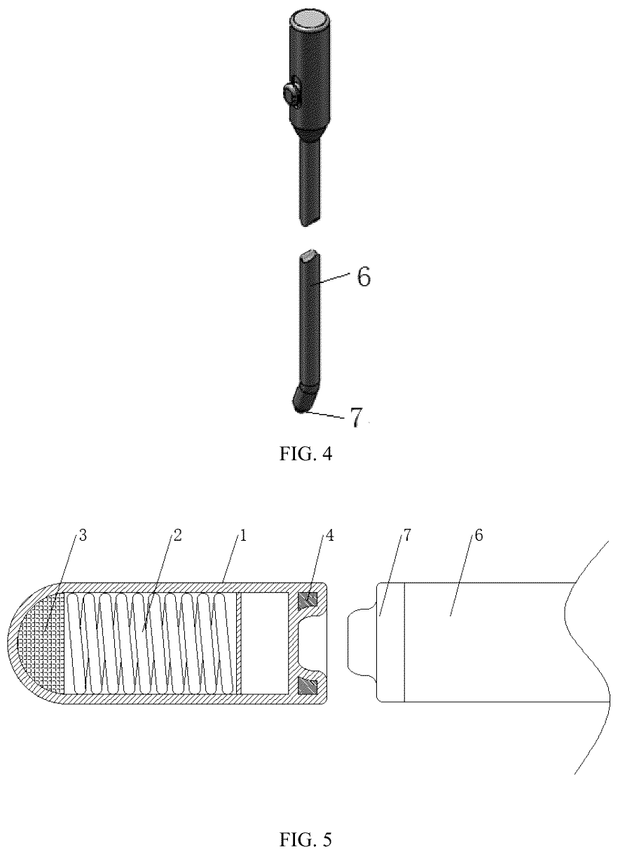

Referring to FIGS. 4 and 5, a placement rod for a intravesical floating hyperthermia device is shown as an embodiment. The intravesical floating hyperthermia device is as described in the embodiment 4. The placement rod includes a rod body 6 and an electromagnet 7 at one end of the rod body 6. The electromagnet 7 engages with the magnet 4 on the floating device for secure handling during transurethral insertion and retrieval. The rod body 6 may include a docking head matching the docking slot on the housing 1, ensuring proper alignment. The electromagnet 7 is configured to, when energized, attract the magnet 4. Through this magnetic attraction and fixation, the rod body 6 can conveniently move and insert the wireless intravesical floating hyperthermia device.

One end of the rod body 6 is provided with a docking head that matches the docking slot at the tail end of the housing 1. The purpose of designing the docking head and the docking slot is to serve as a guide, ensuring the end of the rod body 6 correctly corresponds to the tail end of the housing 1, facilitating subsequent adsorption by the electromagnet 7. The electromagnet 7 is disposed on this docking head.

The rod body 6 is provided with a corresponding power source and a switch at the end away from the docking head. The switch controls the turning on and off of the electromagnet 7, achieving fixation and separation of the intravesical floating hyperthermia device by the rod body 6. This enables the rod body 6 to be easily moved and inserted into the floating device. The placement rod can be used to deliver the intravesical floating hyperthermia device into the bladder via the urethra, or into different parts such as the human intestinal tract or female reproductive tract for tumor hyperthermia.

Furthermore, to further facilitate the magnetic attraction and fixation between the placement rod and the housing 1, the position of the gas chamber can be designed such that the average density of the end of the intravesical floating hyperthermia device proximate to the magnet 4 is slightly lower, and the average density of the opposing end is slightly higher. For example, when the intravesical floating hyperthermia device is applied in the bladder, this design causes the end with the magnet 4 to face downward during flotation, facilitating docking with the placement rod entering through the urethra.

### Embodiment 8

A wireless tumor hyperthermia assembly is shown as an embodiment. The assembly includes the tumor pulse hyperthermia system as described in the embodiment 6 and the placement rod as described in the embodiment 7. The placement rod is used to implant the floating device into the target body cavity (e.g., via the urethra into the bladder). After transurethral insertion, the placement rod is withdrawn from the human body, and the pulse heating apparatus is used to heat the floating device to the set temperature. During each hyperthermia session, hyperthermia can be achieved by wearing a pulse heating device, eliminating the need to repeatedly insert the floating device into the body as in traditional methods, thus avoiding causing significant pain to the patient. The floating device may be designed for single use (e.g., 18 mm length, 6 mm diameter) to prevent contamination.

The disclosed system allows multiple, timed, and temperature-controlled tumor hyperthermia sessions with a single transurethral insertion, eliminating the need for repeated invasive procedures. Throughout the entire treatment process, only two human transurethral insertion procedures are required (initial transurethral insertion and final retrieval). Patients can perform hyperthermia sessions autonomously outside clinical settings, reducing medical resource usage and treatment costs. The built-in camera enables real-time endoscopic examination, enhancing monitoring and diagnostic capabilities. This effectively saves medical resources and patient treatment costs. The floating device has a diameter not greater than 8 mm and an overall length not greater than 18 mm. It is compact in size, low in cost, and can be designed for one-time use. It can be disposed of immediately after use to prevent subsequent pollution.

The various technical features of the above-described embodiments can be combined arbitrarily. To maintain conciseness of description, not all possible combinations of the various technical features in the above embodiments have been explicitly described. However, any combination of these technical features shall be considered as falling within the scope of this specification, provided that there is no contradiction in such combinations.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A wireless intelligently controlled system for intravesical tumor hyperthermia, comprising:
a intravesical floating hyperthermia device configured to be implanted into a urinary bladder cavity and to be remotely controlled from outside the human body to perform hyperthermia on a tumor, the floating device comprising:
a hermetically sealed housing made of a non-metallic material;
a metal coil disposed within the housing and configured to inductively couple with an alternating magnetic field generated outside the human body to generate heat for tumor hyperthermia;
a temperature sensor disposed within the housing and configured to detect a temperature within the housing and generate a corresponding temperature signal;
a wireless transmission module disposed within the housing and configured to wirelessly transmit the temperature signal externally; and
a power supply module disposed within the housing and configured to supply power to the temperature sensor and the wireless transmission module; and
a pulse heating apparatus located outside the human body, comprising:
at least one magnetic field generation module configured to emit the alternating magnetic field toward the metal coil; and
a control module configured to receive the temperature signal and control a heating temperature of the metal coil within a set temperature threshold range based on the temperature signal.

2. The system according to claim 1, wherein the interior of the housing is divided into a cavity and a gas chamber configured to provide buoyancy to float the entire floating device within a urine; and wherein the metal coil, the temperature sensor, the wireless transmission module, and the power supply module are all located within the cavity.

3. The system according to claim 2, wherein the cavity is filled with a thermally conductive urine configured to uniformly transfer heat generated by the metal coil to a surface of the housing.

4. The system according to claim 1, wherein the floating device further comprises a camera module disposed on the housing and configured to capture images and video external to the housing; and wherein the wireless transmission module is further configured to wirelessly transmit the images and the video to an external receiving device;
and wherein the power supply module is further configured to supply power to the camera module.

5. The system according to claim 1, wherein the pulse heating apparatus further comprises a fixing member configured to be secured to the human body, the fixing member is structured as a belt, and two magnetic field generation modules are connected to both front and rear sides of the fixing member.

6. The system according to claim 1, wherein the control module is configured to compare a value of the temperature signal with the temperature threshold range and:
control at least one magnetic field generation module to increase an intensity of the alternating magnetic field if the value is less than a minimum value of the temperature threshold range;
control at least one magnetic field generation module to decrease the intensity of the alternating magnetic field if the value is greater than a maximum value of the temperature threshold range; and
control at least one magnetic field generation module to maintain the intensity of the alternating magnetic field if the value is within the temperature threshold range.

7. The system according to claim 1, wherein the pulse heating apparatus further comprises a portable power source configured to supply power to the at least one magnetic field generation module and the control module.

8. The system according to claim 1, wherein an outer surface of the housing is coated with an antibacterial, hydrophilic, anti-adhesion coating.

9. The system according to claim 4, wherein the camera module comprises a camera unit and a light source.

10. The system according to claim 3, wherein the thermally conductive urine is biocompatible heat transfer fluid.

11. The system according to claim 2, wherein the gas chamber is filled with air or an inert gas.

12. The system according to claim 1, wherein the housing is provided with a docking slot at one end, and a magnet is connected within the docking slot.

13. The system according to claim 1, wherein the control module is further configured to set the temperature threshold range via a signal connection with an external mobile device.

14. The system according to claim 1, wherein the metal coil is formed of copper wire wound into a coil structure.

15. The system according to claim 1, wherein the temperature sensor, the wireless transmission module, and the power supply module are integrated together to form an integrated unit within the housing.

16. The system according to claim 12, wherein the floating device is configured such that an average density of an end of the floating device proximate to the magnet is lower than an average density of an opposing end, thereby causing the end with the magnet to orient downward when the floating device is floating in a urine to facilitate docking.

17. The system according to claim 1, wherein the power supply module comprises a lithium battery.

18. A placement rod for a intravesical floating hyperthermia device, comprising:
a rod body; and
an electromagnet disposed at one end of the rod body;
wherein the electromagnet is configured to, when energized, magnetically engage with a magnet disposed on the intravesical floating hyperthermia device to detachably secure the floating device.

19. The placement rod according to claim 18, wherein an end of the rod body is provided with a docking head matching a docking slot of the housing of the floating device, and wherein the electromagnet is disposed on the docking head.

20. A wireless tumor hyperthermia assembly, comprising:
the tumor pulse hyperthermia system according to any one of claims 1 to 17; and
the placement rod according to claim 18.
